(19) United States
(12) Patent Application Publication
(10) Pub. No.: US 2009/0240070 A1
(43) Pub. Date: Sep. 24, 2009

(54) WHITENING DERMATOLOGICAL PREPARATIONS

(75) Inventors: Harumi Kamachi, Chiba (JP); Hirobumi Aoki, Chiba (JP); Yohei Kurata, Kanagawa (JP); Jiro Takata, Fukuoka (JP)

Correspondence Address: SUGHRUE MION, PLLC, 2100 PENNSYLVANIA AVENUE, N.W., SUITE 800, WASHINGTON, DC, 20037, US

(73) Assignee: SHOWA DENKO K.K., Minato-ku, Tokyo (JP)

(21) Appl. No.: 12/278,479

(22) PCT Filed: Feb. 5, 2007
(86) PCT No.: PCT/JP2007/052427

§ 371 Date: Aug. 6, 2008

Related U.S. Application Data
(60) Provisional application No. 60/771,456, filed on Feb. 9, 2006.

(30) Foreign Application Priority Data
Feb. 6, 2006 (JP) 2006-028662

(51) Int. Cl. C07D 311/72

(52) U.S. Cl. 549/410

(57) ABSTRACT

Disclosed is a dermatological preparation that are safe and are excellent in inhibiting and removing skin pigmentation and in inhibiting skin roughness. A dermatological preparation comprising a tocopherol aminoalkylcarboxylate and/or a salt thereof, the tocopherol aminoalkylcarboxylate being represented by Formula (I):

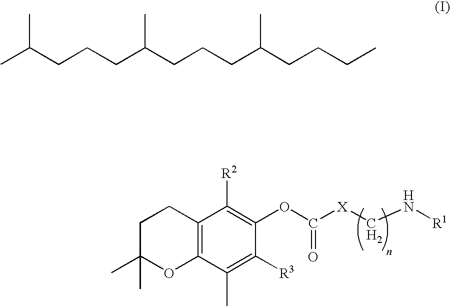

wherein R¹ is a hydrogen atom or a lower alkyl group; R² and R³ are each independently a hydrogen atom or a methyl group; X is a cycloalkylene group of 3 to 6 carbon atoms; and n is 0 or 1.

### CROSS-REFERENCE TO RELATED APPLICATION

This application is an application filed under 35 U.S.C.§111(a) claiming benefit pursuant to 35 U.S.C.§119(e) of the filing date of Provisional Application No. 60/771,456 filed Feb. 9, 2006, pursuant to U.S.C.§111(b).

### FIELD OF THE INVENTION

The present invention relates to dermatological preparations that contain tocopherol aminoalkylcarboxylates and/or salts thereof as active ingredients for inhibiting and removing skin pigmentation. More particularly, the invention relates to dermatological preparations containing tocopherol tranexamates and/or salts thereof.

### BACKGROUND OF THE INVENTION

Dermatological preparations are proposed and used for treating skin pigmentation disorders such as spots and freckles by UV light exposure, and darkening of skin scars, burn scars and surgical scars.

For example, polyphenols are widely known to have depigmentation effects by reducing melanin pigments, and hydroquinones in particular have been clinically used quite often in U.S. and other countries. However, it has been pointed out that these compounds cause strong skin irritation and are incapable of forming dermatological preparations that are safe and free of anxiety.

Tocopherols known as vitamin E (e.g., α-tocopherol, β-tocopherol, γ-tocopherol and δ-tocopherol) and derivatives such as tocopherol acetate and tocopherol nicotinate, have been used in medical drugs and cosmetics on account of their effects of antioxidation, biomembrane stabilization, immune activation and facilitation of blood circulation. Nonpatent Document 1 reports that oral administration of tocopherols is effective against pigmentation. Nonpatent Document 2 reports suppressed pigmentation of cultured cells by a tocopherol derivative, tocopherol ferulate. Patent Document 1 describes that percutaneous administration of tocopherol alkylglycine esters inhibits and removes pigmentation disorders.

Tranexamic acid, which is an aminoalkylcarboxylic acid, and derivatives thereof are reported to have effects of inhibiting pigmentation (Patent Document 2) and of inhibiting skin roughness (Patent Document 3).

As described above, the tocopherols, derivatives thereof, tranexamic acid and derivatives thereof are known to possess effects of inhibiting or removing pigmentation. However, no other compounds are known to be formulated easily in dermatological preparations.

### [Patent Document 1] JP-A-2003-327507

### [Patent Document 2] JP-A-H4-46144

### [Patent Document 3] JP-A-2002-234836

### [Nonpatent Document 1] K. Werininghaus et al., Arch Dermatol, U.S.A., vol. 130, P. 1257, 1997)

### [Nonpatent Document 2] M. Ichihashi et al., Anticancer Res., Greek, vol. 19, p. 3769, 1999)

### DISCLOSURE OF THE INVENTION

In view of the problems as mentioned above, it is an object of the present invention to provide dermatological preparations that are safe and are excellent in inhibiting and removing skin pigmentation and in inhibiting skin roughness.

It is another object of the invention to provide whitening cosmetics having high whitening effects, and pigmentation inhibitors and pigmentation removers each containing the dermatological preparation according to the present invention.

The present inventors studied diligently to achieve the above objects, and have found that tocopherol aminoalkylcarboxylates, particularly tocopherol tranexamates, and salts thereof possess high effects in inhibiting and removing skin pigmentation and in inhibiting skin roughness. The present invention has been completed based on the finding.

The present invention concerns the following.

[1] A dermatological preparation comprising a tocopherol aminoalkylcarboxylate and/or a salt thereof, the tocopherol aminoalkylcarboxylate being represented by Formula (I):

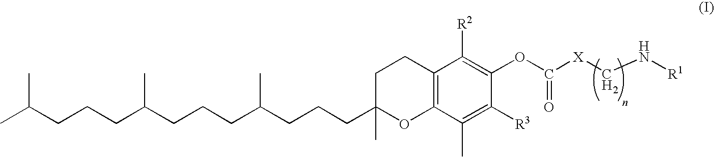

wherein R¹ is a hydrogen atom or a lower alkyl group; R² and R³ are each independently a hydrogen atom or a methyl group; X is a cycloalkylene group of 3 to 6 carbon atoms; and n is 0 or 1.

[2] The dermatological preparation as described in [1],

wherein the tocopherol aminoalkylcarboxylate is at least one tocopherol tranexamate selected from the group consisting of α-tocopherol tranexamate, γ-tocopherol tranexamate and δ-tocopherol tranexamate.

[3] The dermatological preparation as described in [1], wherein the tocopherol aminoalkylcarboxylate is α-tocopherol tranexamate.

[4] The dermatological preparation as described in [3], wherein the α-tocopherol tranexamate is dl-α-tocopherol tranexamate.

[5] The dermatological preparation as described in [3], wherein the α-tocopherol tranexamate is d-α-tocopherol tranexamate.

[6] The dermatological preparation as described in [1], wherein the tocopherol aminoalkylcarboxylate is γ-tocopherol tranexamate.

[7] The dermatological preparation as described in [6], wherein the γ-tocopherol tranexamate is d-γ-tocopherol tranexamate.

[8] The dermatological preparation as described in [1], wherein the tocopherol aminoalkylcarboxylate is δ-tocopherol tranexamate.

[9] The dermatological preparation as described in [8], wherein the δ-tocopherol tranexamate is d-δ-tocopherol tranexamate.

[10] The dermatological preparation as described in [1], wherein the salt of the tocopherol aminoalkylcarboxylate is at least one organic acid salt or inorganic acid salt of a tocopherol tranexamate selected from the group consisting of α-tocopherol tranexamate, γ-tocopherol tranexamate and δ-tocopherol tranexamate.

[11] The dermatological preparation as described in [10], wherein the inorganic acid salt of the tocopherol tranexamate is tocopherol tranexamate hydrochloride.

[12] The dermatological preparation as described in any one of [1] to [11], wherein the dermatological preparation contains the tocopherol aminoalkylcarboxylate and/or the salt thereof in a total amount of 0.1 to 10% by mass.

[13] A whitening cosmetic comprising the dermatological preparation of any one of [1] to [12].

[14] A skin pigmentation inhibitor comprising the dermatological preparation of any one of [1] to [12].

[15] A skin pigmentation remover comprising the dermatological preparation of any one of [1] to [12].

### EFFECTS OF THE INVENTION

The dermatological preparations according to the present invention are safe and are excellent in inhibiting and removing skin pigmentation. Consequently, the dermatological preparations are useful as whitening cosmetics having high whitening effects, and as pigmentation inhibitors and pigmentation removers.

### BRIEF DESCRIPTION OF THE DRAWINGS

FIG. 1 is a picture of a skin model cultured with a standard sample solution as control in Test Example 4;

FIG. 2 is a picture of a skin model cultured with a test article (a) (tranexamic acid) in Test Example 4;

FIG. 3 is a picture of a skin model cultured with a test article (b) (dl-α-tocopherol tranexamate hydrochloride) in Test Example 4; and

FIG. 4 is a picture of a skin model cultured with a test article (c) (dl-γ-tocopherol tranexamate hydrochloride) in Test Example 4.

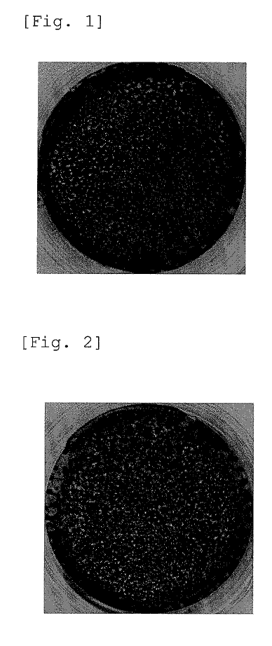

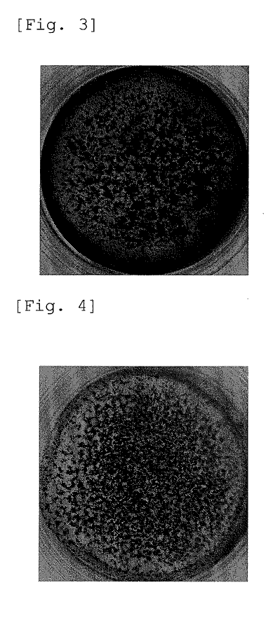

### BEST MODE FOR CARRYING OUT THE INVENTION

The dermatological preparations of the invention contain a specific tocopherol aminoalkylcarboxylate and/or a salt thereof.

The tocopherol aminoalkylcarboxylates and salts thereof will be described.

The tocopherol aminoalkylcarboxylates are represented by Formula (I):

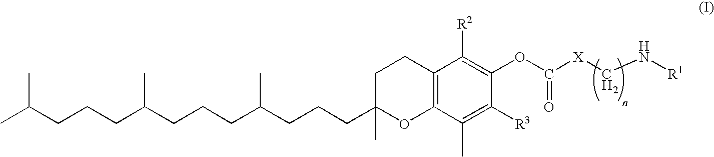

wherein R¹ is a hydrogen atom or a lower alkyl group; R² and R³ are each independently a hydrogen atom or a methyl group; X is a cycloalkylene group of 3 to 6 carbon atoms; and n is 0 or 1.

The lower alkyl group represented by R¹ is preferably a linear or branched alkyl group of 1 to 6 carbon atoms. Examples thereof include methyl, ethyl, n-propyl, n-butyl, isopropyl, isobutyl, 1-methylpropyl, tert-butyl, n-pentyl, 1-ethylpropyl, isoamyl and n-hexyl, with methyl and ethyl being most preferable.

The letter X refers to a cycloalkylene group of 3 to 6 carbon atoms, with examples including cyclopropylene, cyclobutylene, cyclopentylene and cyclohexylene. The cyclohexylene and cyclopentylene represented by Formula (II) and (III), respectively, are preferable.

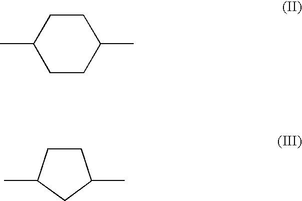

The letter n refers to 0 or 1, preferably 1. When n is 1, the group X is preferably the cyclohexylene group represented by Formula (II), in which case —X—(CH₂)ₙ— is represented by Formula (IV) below:

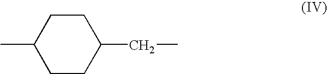

R² and R³ may be simultaneously methyl groups, and in this case the compound represented by Formula (I) is an α-tocopherol derivative. R² may be a hydrogen atom and R³ may be a methyl group, and in this case the compound represented by Formula (I) is a γ-tocopherol derivative. R² and R³ may be simultaneously hydrogen atoms, and in this case the compound represented by Formula (I) is a δ-tocopherol derivative. It is most preferable that R² and R³ be simultaneously methyl groups, that is, the compound represented by Formula (I) be an α-tocopherol derivative.

It is most preferable that the compound used in the invention be an α-tocopherol tranexamate in which R¹ is a hydrogen atom, R² and R³ are methyl groups, and —X—(CH₂)ₙ— is represented by Formula (IV).

The salt of the tocopherol aminoalkylcarboxylate may be an organic acid salt or inorganic acid salt of the compound represented by Formula (I). Preferred examples of the salts include, although not particularly limited to, inorganic acid salts such as hydrohalic acid salts; and organic acid salts such as bile salts, including cholates, glycocholates and deoxycholates. Of the hydrohalic acid salts, hydrochloride is particularly preferable because it is highly soluble in water and its powdery state permits easy handling.

The compound of Formula (I) has an asymmetric carbon atom at the second position of the chroman ring, and therefore a d-, l- or dl-stereoisomer can occur. The compound of Formula (I) may be any of these stereoisomers or may be a combination of two or more of such stereoisomers.

The compound used in the invention may be produced by a known method.

For example, it may be produced by usual esterification of a tocopherol with an aminoalkylcarboxylic acid, a reactive acid derivative thereof or a hydrohalic acid salt of the aminoalkylcarboxylic acid or reactive acid derivative thereof.

The tocopherol is represented by Formula (V):

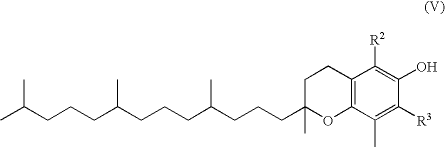

wherein R² and R³ are each independently a hydrogen atom or a methyl group.

The aminoalkylcarboxylic acid is represented by Formula (VI):

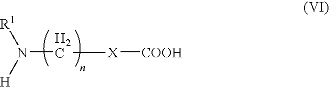

wherein R¹ is a lower alkyl group or a hydrogen atom; X is a cycloalkylene group of 3 to 6 carbon atoms; and n is 0 or 1.

When the esterification is performed using free aminoalkylcarboxylic acid directly, the reaction preferably takes place in the presence of an active esterification reagent such as dicyclohexyl carbodiimide or N,N-disuccinimide oxalate. Pyridine is an optimum solvent for the reaction.

The reactive acid derivative is preferably an acid halide, particularly an acid chloride.

The salt of the tocopherol aminoalkylcarboxylate may be synthesized by adding an acid to the ester produced as described above. The addition of an acid may be performed in the usual manner. Alternatively, it may be produced by the above-described esterification using a salt of aminoalkylcarboxylic acid as a starting material.

The dermatological preparations, whitening cosmetics, skin pigmentation inhibitors and skin pigmentation removers according to the present invention will be described.

The dermatological preparations may be produced by adding the tocopherol aminoalkylcarboxylate and/or the salt thereof, preferably the tocopherol tranexamate and/or the salt thereof to purified water containing ingredients common in dermatological preparations. The total amount of the tocopherol aminoalkylcarboxylate and/or the salt thereof, preferably the tocopherol tranexamate and/or the salt thereof, is 0.1 to 10% by mass, preferably 0.5 to 2% by mass of the dermatological preparation.

Ingredients common in dermatological preparations may be added while still achieving the objects of the present invention. Examples of such ingredients include:

hydrocarbons such as ozokerite, α-olefin oligomers, light isoparaffin, light liquid isoparaffin, squalene, squalane, synthetic squalane, vegetable squalane, ceresin, paraffin, polyethylene powder, polybutene, microcrystalline wax, liquid isoparaffin, liquid paraffin, mineral oil and vaseline;

natural waxes such as jojoba oil, carnauba wax, candelilla wax, rice bran wax, shellac, lanolin, mink oil wax, whale wax, sugarcane wax, sperm oil, beeswax and montan wax, natural fats and oils such as avocado oil, almond oil, olive oil, extra virgin olive oil, sesame oil, rice bran oil, rice oil, rice germ oil, corn oil, soybean oil, maize oil, persic oil, palm kernel oil, palm oil, castor oil, grape seed oil, cotton seed oil, coconut oil, hydrogenated coconut oil, beef tallow, hydrogenated oil, horse oil, mink oil, egg yolk oil, egg yolk fatty oil, rose hip oil, kukui nut oil, evening primrose oil, wheat germ oil, peanut oil, camellia oil, sasanqua oil, cacao butter, Japanese wax, beef bone fat, neatsfoot oil, lard, horse fat, mutton tallow, shea butter, macadamia nut oil and meadowfoam oil;

fatty acids such as lauric acid, myristic acid, palmitic acid, stearic acid, behenic acid, oleic acid, isostearic acid, 12-hydroxystearic acid, undecylenic acid and coconut fatty acid;

higher alcohols such as isostearyl alcohol, octyldodecanol, hexyldecanol, cholesterol, phytosterol, lauryl alcohol, myristyl alcohol, cetanol, stearyl alcohol, oleyl alcohol, behenyl alcohol and cetostearyl alcohol;

alkyl glyceryl ethers such as batyl alcohol, chimyl alcohol, selachyl alcohol and isostearyl glyceryl ether;

esters such as isopropyl myristate, butyl myristate, isopropyl palmitate, ethyl stearate, butyl stearate, ethyl oleate, ethyl linoleate, isopropyl linoleate, cetyl caprylate, hexyl laurate, isooctyl myristate, decyl myristate, myristyl myristate, cetyl myristate, octadecyl myristate, cetyl palmitate, stearyl stearate, decyl oleate, oleyl oleate, cetyl ricinoleate, isostearyl laurate, isotridecyl myristate, isocetyl myristate, isostearyl myristate, octyldodecyl myristate, 2-ethylhexyl palmitate, isocetyl palmitate, isostearyl palmitate, 2-ethylhexyl stearate, isocetyl stearate, isodecyl oleate, octyldodecyl oleate, octyldodecyl ricinoleate, ethyl isostearate, isopropyl isostearate, cetyl 2-ethylhexanoate, cetostearyl 2-ethylhexanoate, stearyl 2-ethylhexanoate, hexyl isostearate, ethylene glycol dioctanoate, ethylene glycol dioleate, propylene glycol dicaprylate, propylene glycol di(caprylate caprate), propylene glycol dicaprate, propylene glycol dioleate, neopentyl glycol dicaprate, neopentyl glycol dioctanoate, glyceryl tricaprylate, glyceryl tri-2-ethylhexanoate, glyceryl tri (caprylate caprate), glyceryl tri(caprylate caprate stearate),

glyceryl triundecylate, glyceryl triisopalmitate, glyceryl triisostearate, trimethylolpropane tri-2-ethylhexanoate, trimethylolpropane triisostearate, pentaerythrityl tetra-2-ethylhexanoate, pentaerythrityl tetramyristate, pentaerythrityl tetraisostearate, diglyceryl tetraisostearate, octyldodecyl neopentanoate, isocetyl octanoate, isostearyl octanoate, 2-ethylhexyl isopelargonate, hexyldecyl dimethyloctanoate, octyldodecyl dimethyloctanoate, 2-ethylhexyl isopalmitate, isocetyl isostearate, isostearyl isostearate, octyldodecyl isostearate, lauryl lactate, myristyl lactate, cetyl lactate, octyldodecyl lactate, triethyl citrate, acetyltriethyl citrate, acetyltributyl citrate, trioctyl citrate, triisocetyl citrate, trioctyldodecyl citrate, diisostearyl malate, 2-ethylhexyl hydroxystearate, di-2-ethylhexyl succinate, diisopropyl adipate, diisobutyl adipate, dioctyl adipate, diheptylundecyl adipate, diethyl sebacate, diisopropyl sebacate, dioctyl sebacate, cholesteryl stearate, cholesteryl isostearate, cholesteryl hydroxystearate, cholesteryl oleate, dihydrocholesteryl oleate, phytosteryl isostearate, phytosteryl oleate,

isocetyl 12-stearoylhydroxystearate, stearyl 12-stearoylhydroxystearate, isostearyl 12-stearoylhydroxystearate, polyoxyethylene (3) polyoxypropylene (1) cetyl ether acetate, polyoxyethylene (3) polyoxypropylene (1) isocetyl ether acetate, isononyl isononanoate, octyl isononanoate, tridecyl isononanoate and isotridecyl isononanoate;

silicone oils such as methyl polysiloxane, methylphenyl polysiloxane, methylhydrogen polysiloxane, methyl cyclopolysiloxane, octamethyl cyclotetrasiloxane, decamethyl cyclopentasiloxane, dodecamethyl cyclohexasiloxane, octamethyl trisiloxane, decamethyl tetrasiloxane, tetradecamethyl hexasiloxane, highly polymerized methyl polysiloxane, dimethyl siloxane/methyl(polyoxyethylene)siloxane/methyl(polyoxypropylene)siloxane copolymer, dimethyl siloxane/methyl(polyoxyethylene)siloxane copolymer, dimethyl siloxane/methyl (polyoxypropylene) siloxane copolymer, dimethyl siloxane/methyl cetyloxysiloxane copolymer, dimethyl siloxane/methyl stearoxysiloxane copolymer, polyether-modified silicones, alcohol-modified silicones, alkyl-modified silicones and amino-modified silicones;

polymers such as sodium alginate, carrageenan, agar, furcelleran, cyamoposis gum, pyrus cyclonia seed, konjac mannan, tamarind gum, tara gum, dextrin, starch, locust bean gum, gum arabic, ghatti gum, karaya gum, tragacanth gum, arabinogalactan, pectin, marmelo, chitosan, curdlan, xanthan gum, gellan gum, cyclodextrin, dextran, pullulan, microcrystalline cellulose, methylcellulose, ethylcellulose, hydroxyethylcellulose, hydroxypropylcellulose, hydroxypropylmethylcellulose, carboxymethylcellulose, carboxy starch, cationized cellulose, starch phosphate, cationized cyamoposis gum, carboxymethyl/hydroxypropylated cyamoposis gum, hydroxypropylated cyamoposis gum, albumin, casein, gelatin, sodium polyacrylate, polyacrylic acid amide, carboxyvinyl polymers, polyethyleneimine, highly polymerized polyethylene glycol, polyvinyl alcohol, polyvinylpyrrolidone, polyvinyl ether, polyacrylamide, acrylic acid polymers, methacrylic acid polymers, maleic acid polymers, vinylpyridine polymers, ethylene/acrylic acid copolymers, vinylpyrrolidone polymers, vinyl alcohol/vinylpyrrolidone copolymers, nitrogen-substituted acrylamide polymers, amino-modified silicones, cationized polymers, dimethylacryl ammonium polymers, acrylic acid-based anionic polymers, methacrylic acid-based anionic polymers, modified silicones, alkyl(C₁₀₋₃₀) acrylate or methacrylate copolymers and polyoxyethylene/polyoxypropylene copolymer;

monoalcohols such as ethanol, isopropyl alcohol, 1-butanol, 2-butanol and benzyl alcohol;

polyhydric alcohols such as ethylene glycol, diethylene glycol, polyethylene glycol, propylene glycol, polypropylene glycol, glycerol, diglycerol, polyglycerol, 1,3-butanediol, triethylene glycol, dipropylene glycol, 3-methyl-1,3-butanediol, 1,2-pentanediol, 1,4-pentanediol, 1,5-pentanediol, 2,4-pentanediol, 2-methyl-2,4-pentanediol, 3-methyl-1,5-pentanediol, 1,2-hexanediol and 1,6-hexanediol;

anionic surfactants such as potassium coconut fatty acid ester, sodium coconut fatty acid ester, triethanolamine coconut fatty acid ester, potassium laurate, sodium laurate, triethanolamine laurate, potassium myristate, sodium myristate, isopropanolamine myristate, potassium palmitate, sodium palmitate, isopropanolamine palmitate, potassium stearate, sodium stearate, triethanolamine stearate, potassium oleate, sodium oleate, sodium castor oil fatty acid ester, zinc undecylenate, zinc laurate, zinc myristate, magnesium myristate, zinc palmitate, zinc stearate, calcium stearate, magnesium stearate, aluminum stearate, calcium myristate, magnesium myristate, aluminum dimyristate, aluminum isostearate, polyoxyethylene laurylether acetic acid, sodium polyoxyethylene laurylether acetate, polyoxyethylene tridecylether acetic acid, sodium polyoxyethylene tridecylether acetate, sodium stearoyl lactate, sodium isostearoyl lactate, lauroylsarcosine sodium, sarcosine coconut fatty acid ester, sarcosine sodium coconut fatty acid ester, sarcosine triethanolamine coconut fatty acid ester,

lauroyl sarcosine, lauroyl sarcosine potassium, lauroyl sarcosine triethanolamine, oleoyl sarcosine, myristoyl sarcosine sodium, sodium stearoyl glutamate, coconut fatty acid acylglutamic acid, coconut fatty acid potassium acylglutamate, coconut fatty acid sodium acylglutamate, coconut fatty acid triethanolamine acylglutamate, lauroyl acylglutamic acid, potassium lauroyl acylglutamate, sodium lauroyl acylglutamate, triethanolamine lauroyl acylglutamate, myristoyl acylglutamic acid, potassium myristoyl acylglutamate, sodium myristoyl acylglutamate, stearoyl acylglutamic acid, potassium stearoyl acylglutamate, disodium stearoyl acylglutamate, hydrogenated tallow fatty acid sodium acylglutamate, coconut fatty acid/hydrogenated tallow fatty acid sodium acylglutamate, methylalanine sodium coconut fatty acid ester, lauroyl methylalanine, lauroyl methylalanine sodium, lauroyl methylalanine triethanolamine, myristoyl methylalanine sodium, lauroyl methyltaurine sodium, methyltaurine potassium coconut fatty acid ester, methyltaurine sodium coconut fatty acid ester, methyltaurine magnesium coconut fatty acid ester, myristoyl methyltaurine sodium, palmitoyl methyltaurine sodium, stearoyl methyltaurine sodium, oleoyl methyltaurine sodium, sodium alkanesulfonate, sodium tetradecenesulfonate, dioctylsodium sulfosuccinate, lauryl disodium sulfosuccinate, ethyl coconut fatty acid ester sodium sulfonate,

sodium laurylsulfate, triethanolamine laurylsulfate, sodium cetyl sulfate, triethanolamine alkyl (11, 13, 15) sulfates, sodium alkyl (12, 13) sulfates, triethanolamine alkyl (12, 13) sulfates, ammonium alkyl (12, 14, 16) sulfates, diethanolamine alkyl (12, 13) sulfates, triethanolamine alkyl (12-14) sulfates, triethanolamine alkyl (12-15) sulfates, magnesium triethanolamine cocoalkylsulfate, ammonium laurylsulfate, potassium laurylsulfate, magnesium laurylsulfate, monoethanolamine laurylsulfate, diethanolamine laurylsulfate, sodium myristylsulfate, sodium stearylsulfate, sodium oleylsulfate, triethanolamine oleylsulfate, sodium polyoxyethylene laurylether sulfate, triethanolamine polyoxyethylene laurylether sulfate, sodium polyoxyethylene (1) alkyl (11, 13, 15) ether sulfate, triethanolamine polyoxyethylene (1) alkyl (11, 13, 15) ether sulfate, sodium polyoxyethylene (3) alkyl (11-15) ether sulfate, sodium polyoxyethylene (2) alkyl (12, 13) ether sulfate,

sodium polyoxyethylene (3) alkyl (12-14) ether sulfate, sodium polyoxyethylene (3) alkyl (12-15) ether sulfate, sodium polyoxyethylene (2) laurylether sulfate, sodium polyoxyethylene (3) myristylether sulfate, higher fatty acid alkanolamide sulfate sodium, laurylphosphoric acid, sodium laurylphosphate, potassium cetylphosphate, diethanolamine cetylphosphate, polyoxyethylene oleylether phosphoric acid, polyoxyethylene laurylether phosphoric acid, sodium polyoxyethylene laurylether phosphate, polyoxyethylene cetylether phosphoric acid, sodium polyoxyethylene cetylether phosphate, polyoxyethylene stearylether phosphoric acid, sodium polyoxyethylene oleylether phosphate, polyoxyethylene alkylphenyl ether phosphoric acid, sodium polyoxyethylene alkylphenyl ether phosphate, triethanolamine polyoxyethylene alkylphenyl ether phosphate, polyoxyethylene octylether phosphoric acid, polyoxyethylene (10) alkyl (12, 13) ether phosphoric acid, polyoxyethylene alkyl (12-15) ether phosphoric acid, polyoxyethylene alkyl (12-16) ether phosphoric acid, triethanolamine polyoxyethylene laurylether phosphate and diethanolamine polyoxyethylene oleylether phosphate;

cationic surfactants such as dioctylamine, dimethylstearylamine, trilaurylamine, stearic acid diethylaminoethylamide, lauryltrimethylammonium chloride, cetyltrimethylammonium chloride, cetyltrimethylammonium bromide, cetyltrimethylammonium saccharin, stearyltrimethylammonium chloride, alkyl (20-22) trimethylammonium chloride, lauryltrimethylammonium bromide, alkyl (16, 18) trimethylammonium chloride, stearyltrimethylammonium bromide, stearyltrimethylammonium saccharin, alkyl (28) trimethylammonium chloride, di(polyoxyethylene)oleylmethylammonium chloride (2EO), dipolyoxyethylenestearylmethylammonium chloride, polyoxyethylene (1) polyoxypropylene (25) diethylmethylammonium chloride, tri(polyoxyethylene)stearylammonium chloride (5EO), distearyldimethylammonium chloride, dialkyl (12-15) dimethylammonium chloride,

dialkyl (12-18) dimethylammonium chloride, dialkyl (14-18) dimethylammonium chloride, dicocoyldimethylammonium chloride, dicetyldimethylammonium chloride, isostearyllauryldimethylammonium chloride, benzalkonium chloride, myristyldimethylbenzylammonium chloride, lauryldimethyl(ethylbenzyl)ammonium chloride, stearyldimethylbenzylammonium chloride, laurylpyridinium chloride, cetylpyridinium chloride, lauroylcolaminoformylmethylpyridinium chloride, stearoylcolaminoformylmethylpyridinium chloride, alkylisoquinolium bromide, methylbenzethonium chloride and benzethonium chloride;

amphoteric surfactants such as 2-alkyl-N-carboxymethyl-N-hydroxyethyl imidazolinium betaine, alkyldiaminoethylglycine hydrochloride, lauryldiaminoethylglycine sodium, undecylhydroxyethylimidazolium betaine sodium, undecyl-N-carboxymethylimidazolium betaine, acyl-N-carboxyethyl-N-hydroxyethylethylenediamine disodium coconut fatty acid ester, acyl-N-carboxyethoxyethyl-N-carboxyethylethylenediamine disodium coconut fatty acid ester, acyl-N-carboxymethoxyethyl-N-carboxymethylethylenediamine disodium coconut fatty acid ester, sodium laurylaminopropionate, sodium laurylaminodipropionate, triethanolamine laurylaminopropionate, acyl-N-carboxyethyl-N-hydroxyethylethylenediamine sodium palm oil fatty acid ester, betaine lauryldimethylaminoacetate, betaine coconut oil alkyldimethylaminoacetate, betaine stearyldimethylaminoacetate, stearyldimethyl betaine sodium, amidopropylbetaine coconut fatty acid ester, amidopropylbetaine palm oil fatty acid ester, lauric acid amide betaine propylacetate, ricinoleic acid amide propylbetaine, stearyldihydroxyethyl betaine and laurylhydroxysulfobetaine;

nonionic surfactants such as polyoxyethylene (10) alkyl (12, 13) ether, polyoxyethylene lauryl ether, polyoxyethylene cetyl ether, polyoxyethylene stearyl ether, polyoxyethylene oleyl ether, polyoxyethylene (3, 7, 12) alkyl (12-14) ether, polyoxyethylene tridecyl ether, polyoxyethylene myristyl ether, polyoxyethylene-sec-alkyl (14) ether, polyoxyethylene isocetyl ether, polyoxyethylene cetostearyl ether, polyoxyethylene (2, 10, 20) isostearyl ether, polyoxyethylene oleylcetyl ether, polyoxyethylene (20) arachyl ether, polyoxyethylene octyldodecyl ether, polyoxyethylene behenyl ether, polyoxyethylene octylphenyl ether, polyoxyethylene nonylphenyl ether, polyoxyethylene dinonylphenyl ether, polyoxyethylene (1) polyoxypropylene (1, 2, 4, 8) cetyl ether, polyoxyethylene (5) polyoxypropylene (1, 2, 4, 8) cetyl ether, polyoxyethylene (10) polyoxypropylene (1, 2, 4, 8) cetyl ether, polyoxyethylene (20) polyoxypropylene (1, 2, 4, 8) cetyl ether, polyoxyethylene polyoxypropylene lauryl ether, polyoxyethylene (3) polyoxypropylene (34) stearyl ether,

polyoxyethylene (4) polyoxypropylene (30) stearyl ether, polyoxyethylene (34) polyoxypropylene (23) stearyl ether, polyoxyethylene polyoxypropylene cetyl ether, polyoxyethylene polyoxypropylene decyltetradecyl ether, polyethylene glycol monolaurate, ethylene glycol monostearate, polyethylene glycol monostearate, polyethylene glycol monooleate, ethylene glycol fatty acid ester, self-emulsifiable ethylene glycol monostearate, diethylene glycol laurate, polyethylene glycol myristate, polyethylene glycol palmitate, diethylene glycol stearate, self-emulsifiable polyethylene glycol (2) monostearate, polyethylene glycol isostearate, ethylene glycol dioctanoate, diethylene glycol dilaurate, polyethylene glycol dilaurate, polyethylene glycol (150) dipalmitate, ethylene glycol distearate, diethylene glycol distearate, polyethylene glycol distearate, ethylene glycol dioleate, polyethylene glycol dioleate, polyethylene glycol diricinoleate, polyoxyethylene (20) sorbitan monolaurate, polyoxyethylene (20) sorbitan monopalmitate, polyoxyethylene (6) sorbitan monostearate, polyoxyethylene (20) sorbitan monostearate, polyoxyethylene (20) sorbitan tristearate, polyoxyethylene (6) sorbitan monooleate, polyoxyethylene (20) sorbitan monooleate, polyoxyethylene (20) sorbitan trioleate,

sorbitan polyoxyethylene (20) coconut fatty acid ester, polyoxyethylene (10-80) sorbitan monolaurate, polyoxyethylene sorbitan tristearate, polyoxyethylene (20) sorbitan isostearate, polyoxyethylene (150) sorbitan tristearate, polyoxyethylene castor oil, polyoxyethylene hydrogenated castor oil, polyoxyethylene (10) hydrogenated castor oil, polyoxyethylene (20) hydrogenated castor oil, polyoxyethylene (40) hydrogenated castor oil, polyoxyethylene (50) hydrogenated castor oil, polyoxyethylene (60) hydrogenated castor oil, lipophilic glyceryl monostearate, lipophilic glyceryl monooleate, self-emulsifiable glyceryl monostearate, glyceryl coconut fatty acid ester, glyceryl laurate, glyceryl myristate, glyceryl isostearate, glyceryl ricinoleate, glyceryl monohydroxystearate, glyceryl oleate, glyceryl linoleate, glyceryl erucate, glyceryl behenate, wheat germ fatty acid glyceride, glyceryl safflower oil fatty acid ester, glyceryl hydrogenated soybean fatty acid ester, saturated fatty acid glyceride, glyceryl cotton seed oil fatty acid, monoisostearic acid glyceryl monomyristate, monotallow fatty acid glyceride, monoglyceryl lanolin fatty acid ester, glyceryl sesquioleate, glyceryl distearate, glyceryl diisostearate, glyceryl diarachidate, sorbitan monolaurate, sorbitan monopalmitate, sorbitan monostearate, sorbitan monoisostearate, sorbitan monooleate, sorbitan sesquistearate, sorbitan sesquioleate, sorbitan tristearate, sorbitan trioleate, sorbitan coconut fatty acid ester, sorbitan isostearate, sorbitan sesquiisostearate, sorbitan distearate, diglyceryl isopalmitate, poly (4-10) glyceryl monolaurate, poly (10) glyceryl monomyristate, poly (2-10) glyceryl monostearate, poly (2-10) glyceryl monoisostearate, poly (2-10) glyceryl monooleate, diglyceryl sesquioleate, poly (2-10) glyceryl diisostearate, poly (6-10) glyceryl distearate, diglyceryl triisostearate,

poly (10) glyceryl tristearate, poly (10) glyceryl trioleate, poly (2) glyceryl tetraisostearate, decaglyceryl pentastearate, poly (6-10) glyceryl pentaoleate, poly (10) glyceryl heptastearate, decaglyceryl decastearate, poly (10) glyceryl decaoleate, condensed poly (6) glyceryl ricinoleate, sucrose fatty acid ester, sucrose coconut fatty acid ester, alkyl glucoside, coconut oil alkyldimethylamine oxide, lauryldimethylamine oxide, dihydroxyethyllauryldimethylamine oxide, stearyldimethylamine oxide, oleyldimethylamine oxide and polyoxyethylene coconut oil alkyldimethylamine oxide;

natural surfactants such as saponin, lecithin, soybean phospholipid, hydrogenated soybean phospholipid, soybean lysophospholipid, hydrogenated soybean lysophospholipid, egg yolk lecithin, hydrogenated egg yolk lysophosphatidylcholine, phosphatidylcholine, phosphatidylethanolamine, phosphatidylserine, sphingophospholipid, sphingomyelin, ganglioside, bile acid, cholic acid, deoxycholic acid, sodium cholate, sodium deoxycholate, spiculisporic acid, rhamnolipid, trehalose lipid, sophorolipid and mannosylerythritol lipid;

ultraviolet light absorbers, including paraminobenzoic acid derivatives such as paraminobenzoic acid, ethyl paraminobenzoate, glyceryl paraminobenzoate, amyl paradimethylaminobenzoate and 2-ethylhexyl paradimethylaminobenzoate, cinnamic acid derivatives such as benzyl cinnamate, diparamethoxy cinnamic acid glyceryl mono-2-ethylhexanoate, methyl 2,4-diisopropylcinnamate, ethyl 2,4-diisopropylcinnamate, potassium paramethoxycinnamate, sodium paramethoxycinnamate, isopropyl paramethoxycinnamate, 2-ethylhexyl paramethoxycinnamate, 2-ethoxyethyl paramethoxycinnamate and ethyl paraethoxycinnamate, urocanic acid derivatives such as urocanic acid and ethyl urocanate, benzophenone derivatives such as 2,4-dihydroxybenzophenone, 2,2′,4,4′-tetrahydroxybenzophenone, 2-hydroxy-4-methoxy-5-sulfobenzophenonesodium, 2-hydroxy-4-methoxybenzophenone-5-sulfonic acid, 2-hydroxy-4-methoxybenzophenone, 2,2′-dihydroxy-4,4′-dimethoxybenzophenone and 2,2′-dihydroxy-4,4′-dimethoxy-5-sulfobenzophenonesodium, salicylic acid derivatives such as ethylene glycol salicylate, 2-ethylhexyl salicylate, phenyl salicylate, benzyl salicylate, p-tert-butylphenyl salicylate, homomethyl salicylate and 3,3,5-trimethylcyclohexyl salicylate, 2-(2′-hydroxy-5′-methoxyphenyl)benzotriazole and 4-tert-butyl-4′-methoxybenzoylmethane;

powders and color materials such as kaolin, silicic anhydride, aluminum magnesium silicate, sericite, talc, boron nitride, mica, montmorillonite, hemp cellulose powder, wheat starch, silk powder, cornstarch, nitro dye, azo dye, nitroso dye, triphenylmethane dye, xanthene dye, quinoline dye, anthraquinone dye, indigo dye, pyrene dye, phthalocyanine dye, natural dyes including flavonoid, quinone, porphyrin, water-soluble annatto, squid ink powder, caramel, guaiazulene, gardenia blue, gardenia yellow, cochineal, shikonin, copper chlorophyllin sodium, paprika dye, safflower red, safflower yellow, laccaic acid and riboflavin butyrate, carbon black, yellow iron oxide, black iron oxide, red iron oxide, iron blue, ultramarine blue, zinc oxide, chromium oxide, titanium oxide, black titanium oxide, zirconium oxide, chromium hydroxide, alumina, magnesium oxide, barium sulfate, aluminum hydroxide, calcium carbonate, lithium cobalt titanate, manganese violet and pearl pigment;

plant extracts such as angelica extract, gambir extract, avocado extract, hydrangea extract, gynostemma pentaphyllum extract, althea extract, arnica extract, oil-soluble arnica extract, almond extract, aloe extract, styrax resin extract, nettle extract, orris extract, turmeric curcuma extract, rose fruit extract, echinacea leaf extract, scutellaria root extract, phellodendron bark extract, Japanese coptis rhizome extract, barley extract, okura extract, hypericum extract, oil-soluble hypericum extract, white nettle extract, oil-soluble white nettle extract, restharrow extract, watercress extract, orange flower water, persimmon tannin, pueraria root extract, Japanese valerian extract, cattail extract, chamomile extract, oil-soluble chamomile extract, chamomile water, oat extract, carrot extract, oil-soluble carrot extract, carrot oil, artemisia capillaris extract, glycyrrhiza extract, glycyrrhiza extracted powder, glycyrrhiza flavonoid, cantharis tincture, raspberry extract, kiwi extract, cinchona extract, cucumber extract, apricot kernel extract, quince seed extract, gardenia extract, sasa albo-marginata extract, sophora root extract, walnut shell extract, clematis extract, black sugar extract, chlorella extract, mulberry bark extract, cinnamon bark extract, gentian extract, geranium herb extract, nuphar extract, burdock root extract, oil-soluble burdock root extract, wheat germ extract, hydrolyzed wheat powder, rice bran extract, fermented rice bran extract, comfrey extract, asiasarum root extract, saffron extract, saponaria extract,

oil-soluble salvia extract, crataegus fruit extract, zanthoxylum fruit extract, shiitake extract, shiitake mushroom extracted powder, rehmannia root extract, lithospermum root extract, oil-soluble lithospermum root extract, perilla herb extract, linden extract, oil-soluble linden extract, filipendula extract, peony root extract, job's tears extract, ginger extract, oil-soluble ginger extract, ginger tincture, acorus calamus rhizome extract, birch extract, oil-soluble birch extract, birch sap, honeysuckle extract, horsetail extract, oil-soluble horsetail extract, scordinin, stevia extract, crataegus extract, sambucus extract, juniper extract, yarrow extract, oil-soluble yarrow extract, peppermint extract, sage extract, oil-soluble sage extract, sage water, mallow extract, celery extract, cnidium rhizome extract, cnidium rhizome water, swertia herb extract, soy extract, jujube extract, thyme extract, green tea extract, tea leaf dry distillated solution, tee seed extract, clove extract, citrus unshiu peel extract, camellia extract, centella extract, oil-soluble walnut extract, duke extract, terminalia extract, Japanese angelica root extract, oil-soluble Japanese angelica root extract, Japanese angelica root water, calendula extract, oil-soluble calendula extract, soy milk powder, peach seed extract, bitter orange peel extract, houttuynia extract, tomato extract, tormentilla extract, natto extract, ginseng extract, oil-soluble ginseng extract, garlic extract, wild rose extract, oil-soluble wild rose extract, malt extract, malt root extract,

ophiopogon tuber extract, parsley extract, barley leaf juice concentrate, peppermint distillate, witch hazel distillate, rose extract, pellitory extract, isodonis extract, loquat leaf extract, oil-soluble loquat leaf extract, coltsfoot extract, hoelen extract, butcher broom extract, butcher broom extracted powder, grape extract, grape leaf extract, grape water, hayflower extract, sponge gourd extract, sponge gourd solution, safflower extract, oil-soluble liden extract, liden water, paeonia extract, hop extract, oil-soluble hop extract, pine extract, silybum marianum fruit extract, horse chestnut extract, oil-soluble horse chestnut extract, mukurossi peel extract, balm mint extract, sweet clover extract, peach leaf extract, oil-soluble peach leaf extract, bean sprouts extract, corn flower extract, corn flower water, eucalyptus extract, saxifraga extract, lily extract, coix extract, oil-soluble coix extract, mugwort extract, Japanese mugwort water, lavender extract, lavender water, apple extract, ganoderma extract, lettuce extract, Chinese milk vetch extract, rose water, romanchamomile extract and burnet extract;

amino acids and peptides such as glycine, valine, leucine, isoleucine, serine, threonine, phenylalanine, thyrosin, tryptophan, cystine, cysteine, methionine, hydroxyproline, aspartic acid, asparagine, glutamic acid, glutamine, histidine, γ-aminobutyric acid, DL-pyrrolidonecarboxylic acid, ε-aminocaproic acid, hydrolyzed elastin, water-soluble elastin, hydrolyzed collagen, water-soluble collagen, casein, glutathione, wheat peptide and soybean peptide;

vitamins and vitamin affecters, including vitamin A such as retinol, retinal, retinoic acid, retinol acetate and retinol palmitate, carotenoids such as α-carotene, β-carotene, γ-carotene, δ-carotene, lycopene, zeaxanthin, cryptoxanthin, echinenone and astaxanthin, vitamin B1 such as thiamines, vitamin B2 such as riboflavin, vitamin B6 such as pyridoxine, pyridoxal and pyridoxamine, vitamin B12 such as cyanocobalamin, vitamin C such as folic acids, nicotinic acid, nicotinic acid amide, pantothenic acids, biotins, L-ascorbic acid, sodium L-ascorbate, L-ascorbyl stearate, L-ascorbyl palmitate, L-ascorbyl dipalmitate, L-ascorbyl tetraisopalmitate, disodium L-ascorbate sulfate, L-ascorbyl magnesium phosphate, L-ascorbyl sodium phosphate, ascorbic acid-2-phosphoric acid ester and L-ascorbic acid-2-glucoside, vitamin D such as ergocalciferol and cholecalciferol, vitamin E such as d-α-tocopherol, DL-α-tocopherol, dl-α-tocopherol acetate, dl-α-tocopherol succinate, β-tocopherol, γ-tocopherol and d-δ-tocopherol, ubiquinones, vitamin K, ferulic acid, γ-oryzanol, α-lipoic acid and orotic acid;

antiseptics such as benzoic acid, sodium benzoate, undecylenic acid, salicylic acid, sorbic acid, potassium sorbate, dehydroacetic acid, sodium dehydroacetate, isobutyl paraoxybenzoate, isopropyl paraoxybenzoate, ethyl paraoxybenzoate, butyl paraoxybenzoate, propyl paraoxybenzoate, benzyl paraoxybenzoate, methyl paraoxybenzoate, methyl sodium paraoxybenzoate, phenoxyethanol, photosensitive agent(kankoh-so) No. 101, photosensitive agent(kankoh-so) No. 201 and photosensitive agent(kankoh-so) No. 401;

antioxidants such as butylhydroxyanisole, butylhydroxytoluene, propyl gallate, erythorbic acid, sodium erythorbate, parahydroxyanisole and octyl gallate;

sequestering agents such as trisodium ethylenediaminehydroxyethyltriacetate, edetic acid, disodium edetate, trisodium edetate, tetrasodium edetate, sodium citrate, gluconic acid, phytic acid, sodium polyphosphate and sodium metaphosphate;

moisturizers such as hyaluronic acid, sodium hyaluronate, sodium chondroitinsulfate, sodium lactate, sodium pyrrolidonecarboxylate, betaine, lactic acid bacteria culture solution, yeast extract and ceramide;

antiinflammatory agents such as glycyrrhizinic acid, trisodium glycyrrhizinate, dipotassium glycyrrhizinate, monoammonium glycyrrhizinate, β-glycyrrhetinic acid, glyceryl glycyrrhetinate, stearyl glycyrrhetinate, lysozyme chloride, hydrocortisone and allantoin;

pH adjusters such as sodium hydroxide, potassium hydroxide and triethanolamine;

salts such as sodium chloride, potassium chloride, magnesium chloride and sodium sulfate;

α-hydroxy acids such as citric acid, glycolic acid, tartaric acid and lactic acid;

whitening agents such as arbutin, α-arbutin and placental extract;

essential oils such as angelica oil, ylang ylang oil, elemi oil, matricaria oil, chamomile oil, cardamom oil, calamus oil, galbanum oil, camphor oil, carrot seed oil, clary sage oil, clove oil, cinnamon bark oil, coriander oil, cypress oil, sandalwood oil, cedarwood oil, citronella oil, cinnamon leaf oil, jasmine absolute, juniper berry oil, ginger extract, spearmint oil, sage oil, cedar oil, geranium oil, thyme oil, tea tree oil, nutmeg oil, niaouli oil, neroli oil, pine oil, basil oil, peppermint oil, patchouli oil, palmarosa oil, fennel oil, petitgrain oil, black pepper oil, frankincense oil, vetivert oil, peppermint oil, bergamot oil, benzoin oil, aniba rosaeodora oil, marjoram oil, myrrh oil, melissa oil, eucalyptus oil, ravensara oil, lavandin oil, lavender oil, lindane oil, rose oil, rosewood oil, rosemary oil and lovage oil;

terpenes such as pinene, terpinene, terpinolene, myrcene and longifolene;

perfumes and water.

The dermatological preparations may be in any forms or formulations that are applied directly to skin. In the broad sense, the dermatological preparations include skin milks, skin creams, foundation creams, cold creams, cleansing creams, shaving creams, cleansing foams, skin toners, lotions, packs, lipsticks, rouges, eye shadows, manicures, soaps, body shampoos, hand soaps, shampoos, conditioners, hair tonics, treatment conditioners, hair creams, hair sprays, hair growth tonics, baldness remedies, hairdyes, styling spritz, depilatories, antidandruff agents, toothpastes, denture adhesives, mouthwashes, permanent waving agents, curling agents, styling agents, ointments, adhesive skin patches, taping agents, bath agents, antiperspirants and sunscreen agents. The dermatological preparations may be used regardless of user's gender and age, and may be used for animal skin as well as human skin.

The dermatological preparations of the invention are particularly suited for use as cosmetics.

The whitening cosmetics, skin pigmentation inhibitors and skin pigmentation removers of the invention contain the dermatological preparations, and may further contain cosmetic ingredients selected from the aforesaid ingredients common in dermatological preparations. Furthermore, they may contain existing cosmetic ingredients while still achieving the objects of the invention.

For example, any of the cosmetic ingredients listed in the following documents are employable: The Japanese Standards of Cosmetic Ingredients 2nd edition (edited by Society of Japanese Pharmacopoeia and published by Yakuji Nippo, Ltd. (1984)), The Japanese Cosmetic Ingredients Codex (edited by Ministry of Health and Welfare, Pharmaceutical Examination Division and published by Yakuji Nippo, Ltd. (1993)), Supplement to The Japanese Cosmetic Ingredients Codex (edited by Ministry of Health and Welfare, Pharmaceutical Examination Division and published by Yakuji Nippo, Ltd. (1993)), The Comprehensive Licensing Standards of Cosmetics by Category (edited by Ministry of Health and Welfare, Pharmaceutical Examination Division and published by Yakuji Nippo, Ltd. (1993)), The Japanese Cosmetic Ingredients Codex by Category (edited by Ministry of Health and Welfare, Pharmaceutical Examination Division and published by Yakuji Nippo, Ltd. (1997)), Dictionary of Cosmetic Ingredients (Nikko Chemicals., Co. Ltd. (1991)), and Latest 300 Cosmetic Functional Materials (CMC Publishing Co., Ltd. (2002)).

The whitening cosmetics, skin pigmentation inhibitors and skin pigmentation removers of the invention preferably contain the above-described tocopherol aminoalkylcarboxylate and/or salt thereof in the same amount as in the dermatological preparations.

The dermatological preparations, whitening cosmetics, and skin pigmentation inhibitors and removers may be produced by common methods depending on the formulations, for example by dissolving, mixing or dispersing the aforesaid ingredients in predetermined amounts. The dermatological preparations, whitening cosmetics, and skin pigmentation inhibitors and removers may be in any states such as solid, liquid, semisolid and gas, and may be in any forms including powder, granules, tablets, gels and foams, although not particularly limited thereto.

### EXAMPLES

The present invention will be described by Examples below without limiting the scope of the invention. In Examples, the amounts of the ingredients are expressed in % by mass.

Tranexamic acid and tranexamic acid derivatives were determined by high-performance liquid chromatography (hereinafter, HPLC).

### (Analysis of Tranexamic Acid)

Liquid chromatograph: Shimadzu LC-10 series

### Column: Shodex C18P 4E

Column temperature: 40° C.
Eluting solution: 0.01 M aqueous ammonium acetate solution (pH 5)/acetonitrile=78/22 (V/V)
Flow rate of eluting solution: 0.8 ml/min
Sample pretreatment: Derivatization into AccQ derivative (in accordance with standard use of a Waters reagent)
Amount of sample injected: 10 μl (autosampler)

### Detection: Fluorescence Ex. 295 nm, Em. 350 nm

### (Analysis of Tranexamic Acid Derivative)

Liquid chromatograph: Shimadzu LC-10 series

### Column: Shodex C18M 4D

Column temperature: 40° C.
Eluting solution: Methanol/acetonitrile (7/3 (V/V)) containing 0.02 M acetic acid/0.02 M ammonium acetate
Flow rate of eluting solution: 0.7 ml/min
Amount of sample injected: 10 μl (autosampler)

### Detection: UV 283 nm

### Synthetic Example 1

16 g (0.1 mol) of tranexamic acid was dissolved in 100 ml of a water-dioxane mixture (1:1, v/v), followed by addition of 30 ml of triethylamine. 26 g of di-tert-butyl dicarbonate was gradually added, followed by stirring for 30 minutes at room temperature. The dioxane was evaporated under reduced pressure, and 50 ml of an aqueous sodium hydrogencarbonate solution (0.5N) was added. The mixture was washed with 100 ml of ethyl acetate to remove impurities. To minimize the loss of the objective compound which is soluble in water, the ethyl acetate phase was washed with 50 ml of an aqueous sodium hydrogencarbonate solution to recover the water-soluble compound in the ethyl acetate phase. These aqueous phases were combined, and the pH of the aqueous phase was adjusted to 3 by adding an aqueous citric acid solution (0.5N) with cooling, and sodium chloride was saturated. Extraction was performed three times each with 100 ml of ethyl acetate. The ethyl acetate phase was dehydrated with anhydrous sodium sulfate, and the solvent was evaporated under reduced pressure. The oily residue was crystallized by cooling to give 21.6 g (95% yield) of BOC-tranexamic acid.

4.5 g of BOC-tranexamic acid prepared above, 8.6 g of dl-α-tocopherol and 4.1 g of N,N′-dicyclohexylcarbodiimide were added to 100 ml of anhydrous pyridine, followed by stirring for 24 hours at room temperature. The solvent was evaporated at 50° C. under reduced pressure, and the residue was combined with iso-propyl ether (100 ml×2 times) to extract the soluble fraction. The extract was concentrated and was subjected to separation/purification by silica gel chromatography (developing solution: iso-propyl ether/n-hexane=1:4 (v/v)), resulting in 11.4 g (85% yield) of oily tocopherol BOC-tranexamate.

10 g of tocopherol BOC-tranexamate prepared above was dissolved in a small amount of acetone, and 100 ml of hydrochloric acid dioxane (3N) was added, followed by stirring for 30 minutes. The solvent was evaporated under reduced pressure. The residue was recrystallized with an acetone-methanol mixture, resulting in 9 g (99% yield) of dl-α-tocopherol tranexamate hydrochloride.

### Synthetic Examples 2 to 4

The procedures of Synthetic Example 1 were repeated except that dl-α-tocopherol was replaced by d-α-tocopherol, d-δ-tocopherol or d-γ-tocopherol, resulting in 8.9 g (98% yield) of d-α-tocopherol tranexamate hydrochloride, 8.5 g (96% yield) of d-δ-tocopherol tranexamate hydrochloride, or 7.8 g (90% yield) of d-γ-tocopherol tranexamate hydrochloride, respectively.

### Example 1

### Lotion 1

The following ingredients 1) to 4) were uniformly dispersed and dissolved such that the final concentrations thereof would be as described below. The liquid was added to the ingredient 5) with stirring to produce a lotion 1.

| | |
|---|---|
| 1) dl-α-Tocopherol tranexamate hydrochloride | 2.00 |
| 2) Ethanol | 5.00 |
| 3) Propylene glycol | 5.00 |
| 4) Methyl parahydroxybenzoate | 0.20 |
| 5) Purified water | 87.8 |

### Lotion 2

The following ingredients 1) to 4) were uniformly dispersed and dissolved such that the final concentrations thereof would be as described below. The liquid was added to the ingredient 5) with stirring to produce a lotion 2.

| | |
|---|---|
| 1) d-α-Tocopherol tranexamate hydrochloride | 2.00 |
| 2) Ethanol | 5.00 |
| 3) Propylene glycol | 5.00 |
| 4) Methyl parahydroxybenzoate | 0.20 |
| 5) Purified water | 87.8 |

### Lotion 3

The following ingredients 1) to 4) were uniformly dispersed and dissolved such that the final concentrations thereof would be as described below. The liquid was added to the ingredient 5) with stirring to produce a lotion 3.

| | |
|---|---|
| 1) d-γ-Tocopherol tranexamate hydrochloride | 2.00 |
| 2) Ethanol | 5.00 |
| 3) Propylene glycol | 5.00 |
| 4) Methyl parahydroxybenzoate | 0.20 |
| 5) Purified water | 87.8 |

### Lotion 4

The following ingredients 1) to 4) were uniformly dispersed and dissolved such that the final concentrations thereof would be as described below. The liquid was added to the ingredient 5) with stirring to produce a lotion 4.

| | |
|---|---|
| 1) d-δ-Tocopherol tranexamate hydrochloride | 2.00 |
| 2) Ethanol | 5.00 |
| 3) Propylene glycol | 5.00 |
| 4) Methyl parahydroxybenzoate | 0.20 |
| 5) Purified water | 87.8 |

### Comparative Example 1

### Lotion 5

### Comparative Control

The following ingredients 1) to 4) were uniformly dispersed and dissolved such that the final concentrations thereof would be as described below. The liquid was added to the ingredient 5) with stirring to produce a lotion 5.

| | |
|---|---|
| 1) Sodium ascorbate-2-phosphate | 2.00 |
| 2) Ethanol | 5.00 |
| 3) Propylene glycol | 5.00 |
| 4) Methyl parahydroxybenzoate | 0.20 |
| 5) Purified water | 87.8 |

### Lotion 6

### Negative Control

The following ingredients 1) to 3) were uniformly dispersed and dissolved such that the final concentrations thereof would be as described below. The liquid was added to the ingredient 4) with stirring to produce a lotion 6.

| | |
|---|---|
| 1) Ethanol | 5.00 |
| 2) Propylene glycol | 5.00 |
| 3) Methyl parahydroxybenzoate | 0.20 |
| 4) Purified water | 89.8 |

The lotions prepared in Example 1 and Comparative Example 1 (lotions 1 to 6) were uniform and showed high temporal stability.

### Example 2

### Lotion 7

The following ingredients 1) to 4) were uniformly dispersed and dissolved such that the final concentrations thereof would be as described below. The liquid was added to the ingredient 5) with stirring to produce a lotion 7.

| | |
|---|---|
| 1) dl-α-Tocopherol tranexamate hydrochloride | 0.10 |
| 2) Ethanol | 5.00 |
| 3) Propylene glycol | 5.00 |
| 4) Methyl parahydroxybenzoate | 0.20 |
| 5) Purified water | 89.7 |

### Lotion 8

The following ingredients 1) to 4) were uniformly dispersed and dissolved such that the final concentrations thereof would be as described below. The liquid was added to the ingredient 5) with stirring to produce a lotion 8.

| | |
|---|---|
| 1) d-α-Tocopherol tranexamate hydrochloride | 0.10 |
| 2) Ethanol | 5.00 |
| 3) Propylene glycol | 5.00 |
| 4) Methyl parahydroxybenzoate | 0.20 |
| 5) Purified water | 89.7 |

### Lotion 9

The following ingredients 1) to 4) were uniformly dispersed and dissolved such that the final concentrations thereof would be as described below. The liquid was added to the ingredient 5) with stirring to produce a lotion 9.

| | | |
|---|---|---|
| 1) | d-γ-Tocopherol tranexamate hydrochloride | 0.10 |
| 2) | Ethanol | 5.00 |
| 3) | Propylene glycol | 5.00 |
| 4) | Methyl parahydroxybenzoate | 0.20 |
| 5) | Purified water | 89.7 |

### Lotion 10

The following ingredients 1) to 4) were uniformly dispersed and dissolved such that the final concentrations thereof would be as described below. The liquid was added to the ingredient 5) with stirring to produce a lotion 10.

| | | |
|---|---|---|
| 1) | d-δ-Tocopherol tranexamate hydrochloride | 0.10 |
| 2) | Ethanol | 5.00 |
| 3) | Propylene glycol | 5.00 |
| 4) | Methyl parahydroxybenzoate | 0.20 |
| 5) | Purified water | 89.7 |

### Comparative Example 2

### Lotion 11

### Comparative Control

The following ingredients 1) to 4) were uniformly dispersed and dissolved such that the final concentrations thereof would be as described below. The liquid was added to the ingredient 5) with stirring to produce a lotion 11.

| | | |
|---|---|---|
| 1) | Sodium ascorbate-2-phosphate | 0.10 |
| 2) | Ethanol | 5.00 |
| 3) | Propylene glycol | 5.00 |
| 4) | Methyl parahydroxybenzoate | 0.20 |
| 5) | Purified water | 89.7 |

### Lotion 12

### Negative Control

The following ingredients 1) to 3) were uniformly dispersed and dissolved such that the final concentrations thereof would be as described below. The liquid was added to the ingredient 4) with stirring to produce a lotion 12.

| | | |
|---|---|---|
| 1) | Ethanol | 5.00 |
| 2) | Propylene glycol | 5.00 |
| 3) | Methyl parahydroxybenzoate | 0.20 |
| 4) | Purified water | 89.8 |

The lotions prepared in Example 2 and Comparative Example 2 (lotions 7 to 12) were uniform and showed high temporal stability.

### Example 3

### Gel Composition 1

The following ingredient 1) was uniformly dispersed in the ingredient 2) such that the final concentration thereof would be as described below. The dispersion was added to the ingredient 3) with stirring to produce an objective gel composition 1.

| | | |
|---|---|---|
| 1) | dl-α-Tocopherol tranexamate hydrochloride | 10 |
| 2) | Glycerin | 20 |
| 3) | Octyldodecyl myristate | 70 |

### Gel Composition 2

The following ingredient 1) was uniformly dispersed in the ingredient 2) such that the final concentration thereof would be as described below. The dispersion was added to the ingredient 3) with stirring to produce an objective gel composition 2.

| | | |
|---|---|---|
| 1) | d-α-Tocopherol tranexamate hydrochloride | 10 |
| 2) | Glycerin | 20 |
| 3) | Octyldodecyl myristate | 70 |

### Gel Composition 3

The following ingredient 1) was uniformly dispersed in the ingredient 2) such that the final concentration thereof would be as described below. The dispersion was added to the ingredient 3) with stirring to produce an objective gel composition 3.

| | | |
|---|---|---|
| 1) | d-γ-Tocopherol tranexamate hydrochloride | 10 |
| 2) | Glycerin | 20 |
| 3) | Octyldodecyl myristate | 70 |

### Gel Composition 4

The following ingredient 1) was uniformly dispersed in the ingredient 2) such that the final concentration thereof would be as described below. The dispersion was added to the ingredient 3) with stirring to produce an objective gel composition 4.

| | | |
|---|---|---|
| 1) | d-δ-Tocopherol tranexamate hydrochloride | 10 |
| 2) | Glycerin | 20 |
| 3) | Octyldodecyl myristate | 70 |

### Comparative Example 3

### Gel Composition 5

### Comparative Control

The following ingredient 1) was uniformly dispersed in the ingredient 2) such that the final concentration thereof would be as described below. The dispersion was added to the ingredient 3) with stirring to produce an objective gel composition 5.

| | | |
|---|---|---|
| 1) | Sodium ascorbate-2-phosphate | 10 |
| 2) | Glycerin | 20 |
| 3) | Octyldodecyl myristate | 70 |

### Gel Composition 6

### Negative Control

The following ingredient 1) was uniformly dispersed in the ingredient 2) such that the final concentration thereof would be as described below. The dispersion was added to the ingredient 3) with stirring to produce an objective gel composition 6.

| | | |
|---|---|---|
| 1) | Purified water | 10 |
| 2) | Glycerin | 20 |
| 3) | Octyldodecyl myristate | 70 |

The gel compositions prepared in Example 3 and Comparative Example 3 (gel compositions 1 to 6) were uniform and showed high temporal stability.

### Test Example 1

### Pigmentation Inhibiting Effect

Fifty male guinea pigs (7 weeks old, weiser maple species, SPF) were shorn on their entire backs with electric clippers (0.05 mm blade) and were shaven with an electric shaver. The exposed skin was covered with an adhesive stretch bandage (SILKYTEX, overlaid with aluminum foil on outer surface) which had six windows 1.5 cm×1.5 cm in size.

The lotions 1-12 and the gel compositions 1-6 obtained in Examples 1-3 and Comparative Examples 1-3 were sequentially applied to 10 window openings each in an amount of 0.05 ml.

Four hours later, the treated sites were cleaned with wet absorbent cotton and were dried. The guinea pigs were each secured in a retaining apparatus and were exposed to ultraviolet beams (UVB) of medium wavelength by means of an ultraviolet irradiation device (product of Shinano Seisakusho, provided with fluorescent lamps FL 40S/E30 (TOSHIBA LIGHTING & TECHNOLOGY CORPORATION) and six SE lamps) at a distance of about 10 cm, so that the skin was irradiated with ultraviolet rays in 300 mJ/cm² dose.

The irradiation was followed by application of the same lotions 1-12 and the same gel compositions 1-6 to the same corresponding sites each in an amount of 0.05 ml.

These procedures were repeatedly carried out for 3 days. After 14 days from the final irradiation, the pigmentation degree was visually evaluated by marks according to the following criteria. Separately, the brightness of skin color was measured with a color difference meter (CR-20 available from MINOLTA Co., Ltd.) at five points: the four corners and the center point of the treated/irradiated site.

Pigmentation inhibiting effects were evaluated based on the average of the marks (for the 10 sites) and the average of the brightness (for the 50 points) of the preparation.

### Criteria for Evaluation of Pigmentation Degree

| | |
|---|---|
| No pigmentation | 0 |
| Slight pigmentation | 1 |
| Mild pigmentation | 2 |
| Moderate pigmentation | 3 |
| Severe pigmentation | 4 |

The results are shown in Table 1.

TABLE 1

| | Mark | |
|---|---|---|
| Preparation | (average) | Brightness |
| Lotion 1 | 0.7 | 63.1 |
| Lotion 2 | 0.6 | 62.8 |
| Lotion 3 | 0.5 | 64.0 |
| Lotion 4 | 0.5 | 64.0 |
| Lotion 5 (comparative control) | 2.0 | 61.0 |
| Lotion 6 (negative control) | 3.4 | 57.2 |
| Lotion 7 | 1.4 | 61.9 |
| Lotion 8 | 1.2 | 62.8 |
| Lotion 9 | 1.0 | 62.5 |
| Lotion 10 | 1.1 | 62.6 |
| Lotion 11 (comparative control) | 2.8 | 59.9 |
| Lotion 12 (negative control) | 3.5 | 58.0 |
| Gel composition 1 | 1.8 | 61.3 |
| Gel composition 2 | 1.7 | 61.4 |
| Gel composition 3 | 1.6 | 61.4 |
| Gel composition 4 | 1.7 | 61.4 |
| Gel composition 5 (comparative control) | 3.0 | 60.2 |
| Gel composition 6 (negative control) | 3.6 | 56.9 |

The results in Table 1 prove excellent pigmentation inhibiting effects of the lotions (1-4 and 7-10) and the gel compositions (1-4) according to the present invention.

### Test Example 2

### Pigmentation Removing Effects

Fifty male guinea pigs (6 weeks old, weiser maple species, SPF) were shorn on their entire backs with electric clippers (0.05 mm blade) and were shaven with an electric shaver. The exposed skin was covered with an adhesive stretch bandage (SILKYTEX, overlaid with aluminum foil on outer surface) which had six windows 1.5 cm×1.5 cm in size. The guinea pigs were each secured in a retaining apparatus and were exposed to ultraviolet beams (UVB) of medium wavelength by means of an ultraviolet irradiation device (product of Shinano Seisakusho, provided with fluorescent lamps FL 40S/E30 (TOSHIBA LIGHTING & TECHNOLOGY CORPORATION) and six SE lamps) at a distance of about 10 cm, so that the skin was irradiated with ultraviolet rays in 750 mJ/cm² dose.

From the 4th day to the 28th day after the irradiation, the lotions 1-12 and the gel compositions 1-6 obtained in Examples 1-3 and Comparative Examples 1-3 were sequentially applied to 10 window openings each in an amount of 0.05 ml twice a day in the morning and the evening.

After 28 days after the irradiation, the pigmentation degree was visually evaluated by marks according to the same criteria as in Test Example 1.

Pigmentation removing effects were evaluated based on the average of the marks (for the 10 sites) of the preparation.

The results are shown in Table 2.

TABLE 2

| | Mark |
|---|---|
| Preparation | (average) |
| Lotion 1 | 1.5 |
| Lotion 2 | 1.6 |
| Lotion 3 | 1.7 |
| Lotion 4 | 1.5 |
| Lotion 5 (comparative control) | 3.2 |
| Lotion 6 (negative control) | 3.5 |
| Lotion 7 | 2.8 |
| Lotion 8 | 2.5 |
| Lotion 9 | 2.2 |
| Lotion 10 | 2.7 |
| Lotion 11 (comparative control) | 3.0 |
| Lotion 12 (negative control) | 3.4 |
| Gel composition 1 | 1.8 |
| Gel composition 2 | 1.9 |
| Gel composition 3 | 2.0 |
| Gel composition 4 | 1.7 |
| Gel composition 5 (comparative control) | 3.0 |
| Gel composition 6 (negative control) | 3.6 |

The results in Table 2 prove excellent pigmentation removing effects of the lotions (1-4 and 7-10) and the gel compositions (1-4) according to the present invention.

### Test Example 3

### Skin Penetration Test

### Effects of Preventing Skin Roughness

Tested were:

(a) Tranexamic acid (manufactured by Wako Pure Chemical Industries, Ltd.)

(b) dl-α-Tocopherol tranexamate hydrochloride (Synthetic Example 1)

(c) dl-γ-Tocopherol tranexamate hydrochloride (Synthetic Example 4)

The above test articles, each 0.5% by mass, were dissolved separately in Dulbecco's PBS (−) containing 10% bovine serum albumin to give test article solutions.

2-cm square skin pieces of minipig (Charles River Laboratories Inc. (United States of America)) were placed in the Netwell (manufactured by Corning Incorporated), and assay rings (Teflon® rings with a center opening) on which a silicon sealant had been applied were pressed and fixed to the epidermis of the skin. Subsequently, 2 ml of a skin culture medium (TOYOBO CO., LTD.) was added to a multiwell plate, and the Netwell was set such that skin lower portions would be soaked. Each of the test article solutions, 0.1 ml, was slowly poured into the opening of assay ring. When no leakage was confirmed, the Netwell was sealed with multiplate seals and were allowed to stand in an incubator at 37° C. and 5% carbon dioxide.

After 24 hours, the test article solutions on the skin pieces were pipetted. The skin pieces were recovered from the Netwell, and distilled water in a washing bottle was poured thereover for washing. The assay rings were detached, and the central portions that had contacted with the solution were punched out with an 8 mm biopsy punch. The excised skin pieces were transferred to 1.5 ml tubes, and were washed with distilled water and ethanol alternately several times to remove the test article adsorbed on the skin surface.

Thereafter, the washed skin pieces were fed with 0.5 ml of distilled water, followed by freezing and thawing. They were crushed with a microhomogenizer designed for 1.5 ml tubes and were centrifuged at 12,000 rpm for 5 minutes to separate uncrushed residues. Thus, a tissue extract was obtained.

The test articles in the tissue extracts were determined by HPLC. To determine tranexamic acid, 0.01 ml of a 10% perchloric acid solution was added to 0.04 ml of the extract followed by stirring, and the mixture was centrifuged to obtain a supernatant, which was analyzed. To determine the tranexamic acid derivative, 0.06 ml of a 1 M Tris buffer solution (pH 9) was admixed with 0.04 ml of the extract, and the derivative was extracted using 0.1 ml of ethyl acetate, the solvent phase containing the derivative was analyzed.

The protein in the tissue extracts was determined by the Lowry method.

The Lowry method involved the following reagents that had been prepared with reference to Shin Seikagaku Jikken Kouza 1, Protein 1, p. 85-107.

Reagent 1: 0.1 M sodium hydroxide solution containing 2 wt % sodium carbonate

Reagent 2: 1 wt % sodium citrate solution containing 0.5 wt % copper sulfate pentahydrate

Reagent 3: 1 N phenol reagent

Reagent 4: 50:1 mixture of Reagent 1 and Reagent 2

Standard sample: solution containing 0.1-1.5 mg of bovine serum albumin

The determination procedures were as follows:

400 μl of the reagent 4 was added to a sample tube containing 20 μl of the tissue extract or the standard sample solution, followed by mixing. The mixture was allowed to stand at room temperature for at least 15 minutes. Thereafter, 40 μl of the reagent 3 was added and mixed together, and the mixture was allowed to stand at room temperature for at least 30 minutes. The absorbance at 750 nm was measured with a spectrophotometer, and the protein concentration in the tissue extract was determined using a calibration curve obtained with the standard sample.

Table 3 shows the amounts of tranexamic acid and tranexamic acid derivative in the samples analyzed (unit: μmol/mg skin protein).

TABLE 3

| Sample analyzed | Substance to be | |
|---|---|---|
| (Test article) | determined | Determined value |
| Skin 1 (a) | a | 0.034 |
| Skin 2 (b) | b | 1.4 |
| Skin 3 (c) | c | 1.1 |

Table 3 establishes that more tranexamic acid derivatives (b) and (c) penetrate the skin than tranexamic acid (a) and prevent skin roughness more effectively.

### Test Example 4

### Test of Inhibiting Melanin Synthesis

### Whitening Effects

Tested were:

(a) Tranexamic acid (manufactured by Wako Pure Chemical Industries, Ltd.)

(b) dl-α-Tocopherol tranexamate hydrochloride (Synthetic Example 1)

(c) dl-γ-Tocopherol tranexamate hydrochloride (Synthetic Example 4)

The above test articles, each 0.5% by mass, were dissolved separately in sterilized water containing 10% bovine serum albumin and 1% DMSO to give test article solutions.

0.1 ml of the test article solution was placed in a skin model cup which contained a culture skin model (MEL-300, manufactured by KURABO INDUSTRIES LTD.) The culture skin model contained melanin cells. The skin model was cultured for 11 days using the supplied culture medium LLMM. Each article was tested two times. The culture medium was changed every other day during the incubation.

After 11 days of incubation, the skin model was washed and was measured for the cell survival rate by colorimetry (570 nm) with the Alamar Blue reagent (manufactured by Molecular Probe). Thereafter, the melanin synthesized in the skin model was determined in the following manner.

The skin model was soaked overnight in a liquid which consisted of 0.2 ml of a 10 mM tris buffer solution (pH 6.8) containing 1% SDS and 0.05 mM EDTA, and 0.02 ml of 5 mg/ml Proteinase K. On the next day, 0.02 ml of 5 mg/ml Proteinase K was added, followed by heating at 45° C. for 4 hours. Consequently, the skin model was dissolved. The lysate was made alkaline with 0.025 ml of a 500 mM sodium carbonate solution, and was combined with 5 μl of 30% hydrogen peroxide, followed by heating at 80° C. for 30 minutes to perform reaction. To prepare a standard sample solution, 0.24 ml of a melanin solution of known concentration was combined with 0.025 ml of a 500 mM sodium carbonate solution and 5 μl of 30% hydrogen peroxide, followed by heating at 80° C. for 30 minutes to perform reaction.

The standard sample solution and the lysate obtained were each cooled and extracted with 0.1 ml of chloroform/methanol (2/1). The aqueous phase was measured for the absorbance at 405 nm, and the melanin synthesized was determined using a calibration curve obtained with the standard sample.

Table 4 shows the amounts of melanin in the skin models cultured with the test articles, relative to the control (skin model cultured with the standard sample solution) (100%).

TABLE 4

| | Amount of melanin synthesized |
|---|---|
| Substance added (test article) | (% relative to the control) |
| Control | 100 |
| a | 99 |
| b | 60 |
| c | 44 |

Table 4 establishes that the tranexamic acid derivatives (b) and (c) inhibit the melanin synthesis more effectively than tranexamic acid and provide superior whitening effects.

1. A dermatological preparation comprising a tocopherol aminoalkylcarboxylate and/or a salt thereof, the tocopherol aminoalkylcarboxylate being represented by Formula (I): 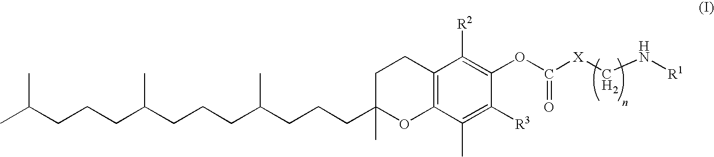
wherein R¹ is a hydrogen atom or a lower alkyl group; R² and R³ are each independently a hydrogen atom or a methyl group; X is a cycloalkylene group of 3 to 6 carbon atoms; and n is 0 or 1.

2. The dermatological preparation according to claim 1, wherein the tocopherol aminoalkylcarboxylate is at least one tocopherol tranexamate selected from the group consisting of α-tocopherol tranexamate, γ-tocopherol tranexamate and δ-tocopherol tranexamate.

3. The dermatological preparation according to claim 1, wherein the tocopherol aminoalkylcarboxylate is α-tocopherol tranexamate.

4. The dermatological preparation according to claim 3, wherein the α-tocopherol tranexamate is dl-α-tocopherol tranexamate.

5. The dermatological preparation according to claim 3, wherein the α-tocopherol tranexamate is d-α-tocopherol tranexamate.

6. The dermatological preparation according to claim 1, wherein the tocopherol aminoalkylcarboxylate is γ-tocopherol tranexamate.

7. The dermatological preparation according to claim 6, wherein the γ-tocopherol tranexamate is d-γ-tocopherol tranexamate.

8. The dermatological preparation according to claim 1, wherein the tocopherol aminoalkylcarboxylate is δ-tocopherol tranexamate.

9. The dermatological preparation according to claim 8, wherein the δ-tocopherol tranexamate is d-δ-tocopherol tranexamate.

10. The dermatological preparation according to claim 1, wherein the salt of the tocopherol aminoalkylcarboxylate is at least one organic acid salt or inorganic acid salt of a tocopherol tranexamate selected from the group consisting of α-tocopherol tranexamate, γ-tocopherol tranexamate and δ-tocopherol tranexamate.

11. The dermatological preparation according to claim 10, wherein the inorganic acid salt of the tocopherol tranexamate is tocopherol tranexamate hydrochloride.

12. The dermatological preparation according to claim 1, wherein the dermatological preparation contains the tocopherol aminoalkylcarboxylate and/or the salt thereof in a total amount of 0.1 to 10% by mass.

13. A whitening cosmetic comprising the dermatological preparation according to claim 1.

14. A skin pigmentation inhibitor comprising the dermatological preparation according to claim 1.

15. A skin pigmentation remover comprising the dermatological preparation according to claim 1.
